(19) United States
(12) Patent Application Publication
(10) Pub. No.: US 2019/0070077 A1
(43) Pub. Date: Mar. 7, 2019

(54) COSMETIC

(71) Applicant: Geol Chemical Co., Ltd., Nara (JP)

(72) Inventors: Keiji MORIMURA, Katsuragi-shi, Nara (JP); Naruko NANGO, Katsuragi-shi, Nara (JP)

(21) Appl. No.: 16/083,648

(22) PCT Filed: Mar. 13, 2017
(86) PCT No.: PCT/JP2017/009975

§ 371 Date: Sep. 10, 2018

(30) Foreign Application Priority Data
Mar. 22, 2016 (JP) 2016-056777

(51) Int. Cl. A61K 8/04; A61Q 1/14; A61K 8/81; A61K 8/34; A61K 8/37; A61Q 19/00

(52) U.S. Cl. CPC A61K 8/042; A61Q 1/14; A61K 8/8152; A61K 2800/262; A61K 8/37; A61Q 19/00; A61K 8/345

(57) ABSTRACT

Provided is a cosmetic that, even when it is not a product having an emulsified composition product on the inner layer and an aqueous gel product on the outer layer (for example, when their formulations are the same), has the functions of both the inner layer product and the outer layer product, that can stably maintain a multiple-layer state in which the inner layer product is enclosed in the outer layer product for a longer time, and that has a good aesthetic appearance. A cosmetic 1 according to the present invention includes a transparent or semitransparent container 2, a transparent or semitransparent product A that fills the container 2, and a product B that is embedded in the product A. The product A and the product B have the same formulation, and the product A or the product B contains a colloid. Preferably, the cosmetic 1 according to the present invention is formed of a plurality of products having the same formulation, has the functions of the plurality of products, can maintain the multiple-layer state for a long time, and has a good aesthetic appearance.

### TECHNICAL FIELD

The present invention relates to a cosmetic product in which a gel-like, cream-like, or semisolid formulation is loaded into a container wherein a colloid is included inside of the formulation and inside of the cosmetic product stably includes a formulation in a predetermined shape.

### BACKGROUND ART

There is a demand for a product that has multiple functionalities as a single product for a skin care product with various functionalities such as cream, milky lotion, lotion, beauty location, beauty gel, beauty oil, cleansing gel, cleansing cream, cleansing milky lotion, cleansing oil, packs, and cleansing foam. Cosmetic products are often commercially sold with a formulation loaded in a container that incorporates various designs. Due to the nature of the product, the outward appearance, i.e., aesthetics, leads to significant motivation for purchase. For this reason, development of products with superb aesthetics, while improving the functionality of the cosmetic products, is ongoing.

For example, the Applicant has previously proposed cosmetic products that can inhibit an inner layer formulation from mixing with an outer layer formulation at an interface over time to maintain stability by using formulations with different compositions between the inner layer and the outer side (see Patent Literature 1).

From the viewpoint of obtaining a stable multilayer transparent gel composition with superb outward appearance, a multilayer transparent gel composition has been proposed, which has been adjusted to have a specific viscosity gradient by adding a solidification agent to an outer layer composition and an inner layer composition constituted of the same base (see Patent Literature 2).

### CITATION LIST

### Patent Literature

[PTL 1] Japanese Laid-Open Publication No. 2011-6374

[PTL 2] Japanese Laid-Open Publication No. 2005-89337

### SUMMARY OF INVENTION

### Technical Problem

However, when additional tests were conducted using the multilayer transparent gel composition described in Patent Literature 2, it was found that an inner layer composition cannot be loaded into an outer layer composition in some cases due to the high viscosity of the compositions depending on the combination of the outer layer composition and inner layer composition. It was also found that if the temperature condition for loading the inner layer composition is increased, a desired shape could not be attained, or liquefied outer layer composition and inner layer composition mixed with each other and solidified, so that formation/maintenance of a multilayer state and aesthetics could not be achieved simultaneously.

Meanwhile, use of the technique of Patent Literature 1 enables highly stable and reproducible loading even under room temperature conditions, while allowing the inner layer to maintain a desired shape even after loading, but when formulations other than those using an emulsified composition as the inner layer and aqueous gel as the outer layer were used (e.g., when combining gel with gel), the formulation in the inner layer gradually seeped out to the outer layer with the passage of time in some cases. For this reason, there were further grounds for improvement from the viewpoint of retaining the shape of the inner layer formulation over a long period of time and inhibiting the mixture of formulations at an interface even when the dosage form is the same.

The present invention has been conceived in view of the above circumstances. The objective of the present invention is to provide a novel cosmetic product, which has functionalities of both the inner layer formulation and the outer layer formulation, is capable of stably retaining a multilayer state where the inner layer formulation is included inside the outer layer formulation for a longer period of time, and has superb aesthetics, even for formulations other than those using an emulsified composition as the inner layer and aqueous gel as the outer layer (e.g., when combining gel with gel).

### Solution to Problem

As a result of diligent research to achieve the objective, the inventors have discovered that a cosmetic product, which is capable of stably retaining a multilayer state where the inner layer formulation is included inside the outer layer formulation for a longer period of time and has superb aesthetics, even for formulations other than those using an emulsified composition as the inner layer and aqueous gel as the outer layer (e.g., formulations having the same dosage form) can be provided by adding a colloid as a component of the formulation to complete the present invention.

In other words, the present invention encompasses the following embodiments.

### (Item 1)

A cosmetic product having a transparent or semitransparent container, a transparent or semitransparent formulation A loaded into the container, and a formulation B embedded in the formulation A,

wherein at least one of the formulations A and B comprise a colloid.

### (Item 2)

The cosmetic product of item 1, wherein the formulation A and the formulation B have the same dosage form.

### (Item 3)

The cosmetic product of item 2, wherein the dosage form of the formulation A and the formulation B is aqueous gel.

### (Item 4)

The cosmetic product of any one of items 1 to 3, wherein the formulation B is transparent or semitransparent.

### (Item 5)

The cosmetic product of any one of items 1 to 4, wherein the formulation B comprises a colloid, and an amount of a colloid dispersion added relative to the entire amount of the formulation B is 0.001 to 50.00% by weight.

### (Item 6)

The cosmetic product of any one of items 1 to 5, wherein an average particle size of the colloid is 1 nm to 10 μm.

### (Item 7)

The cosmetic product of any one of items 1 to 6, wherein the colloid is a metal colloid.

### (Item 8)

The cosmetic product of item 7, wherein the metal colloid is a metal colloid selected from the group consisting of platinum, gold, silver, palladium, rhodium, ruthenium, iridium, and a combination thereof, and an alloy prepared by mixing these metals.

### (Item 9)

The cosmetic product of any one of items 1 to 8, wherein a maximum load indicating a viscosity of the formulation A and the formulation B measured under the following conditions is 0.2 N to 2.0 N:

a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

### (Item 10)

The cosmetic product of any one of items 1 to 9, wherein a ratio of the formulation B to the entire amount of a formulation is 10 to 60% by weight.

### (Item 11)

The cosmetic product of any one of items 1 to 10, wherein a shape of the formulation B is a shape selected from the group consisting of a perfectly spherical shape, a spherical shape, an egg shape, a spheroid shape, a shape with an oval cross-section, a perfectly spherical shape with a protrusion on a surface, a spherical shape with a protrusion on a surface, an egg shape with a protrusion on a surface, a spheroid shape with a protrusion on a surface, and a shape with an oval cross-section with a protrusion on a surface.

### Advantageous Effects of Invention

The present invention provides a novel cosmetic product, which has functionalities of both an inner layer formulation and outer layer formulation, is capable of stably retaining a multilayer state where the inner layer formulation is included inside the outer layer formulation for a longer period of time, and has superb aesthetics, even for formulations other than those using an emulsified composition as the inner layer and aqueous gel as the outer layer (e.g., formulations with the same dosage form).

### BRIEF DESCRIPTION OF DRAWINGS

FIG. 1 shows (a) to (d), which are schematic diagrams depicting various forms of the cosmetic product according to one embodiment of the present invention.

FIG. 2 shows photograph images showing the state of formulations for a product loaded with two formulations, in which the inner layer gel of prescription 2 is loaded into the outer layer gel of prescription 1 in the Examples, (a) immediately after loading, (b) after 6 hours from loading, (c) after 24 hours from loading, and (d) after 48 hours from loading the inner layer gel.

FIG. 3 shows photograph images showing the state of formulations for a product loaded with two formulations, in which the inner layer gel of prescription 3 is loaded into the outer layer gel of prescription 1 in the Examples, (a) immediately after loading, (b) after 6 hours from loading, (c) after 24 hours from loading, and (d) after 48 hours from loading the inner layer gel.

FIG. 4 shows photograph images showing the state of formulations for a product loaded with two formulations, in which the inner layer gel of prescription 4 is loaded into the outer layer gel of prescription 1 in the Examples, (a) immediately after loading, (b) after 6 hours from loading, (c) after 24 hours from loading, and (d) after 48 hours from loading the inner layer gel.

FIG. 5 shows photograph images showing the state of formulations for a product loaded with two formulations, in which the inner layer gel of prescription 5 is loaded into the outer layer gel of prescription 1 in the Examples, (a) immediately after loading, (b) after 6 hours from loading, (c) after 24 hours from loading, and (d) after 48 hours from loading the inner layer gel.

FIG. 6 shows photograph images showing the state of formulations for a product loaded with two formulations, in which the inner layer gel of prescription 6 is loaded into the outer layer gel of prescription 1 in the Examples, (a) immediately after loading, (b) after 6 hours from loading, (c) after 24 hours from loading, and (d) after 48 hours from loading the inner layer gel.

FIG. 7 shows photograph images showing the state of formulations for a product loaded with two formulations, in which the inner layer gel of prescription 7 is loaded into the outer layer gel of prescription 1 in the Examples, (a) immediately after loading, (b) after 6 hours from loading, (c) after 24 hours from loading, and (d) after 48 hours from loading the inner layer gel.

FIG. 8 shows photograph images showing the state of formulations for a product loaded with two formulations, in which the inner layer gel of prescription 8 is loaded into the outer layer gel of prescription 1 in the Examples, (a) immediately after loading, (b) after 6 hours from loading, (c) after 24 hours from loading, and (d) after 48 hours from loading the inner layer gel.

FIG. 9 shows photograph images showing the state of formulations for a product loaded with two formulations, in which the inner layer gel of prescription 9 is loaded into the outer layer gel of prescription 1 in the Examples, (a) immediately after loading, (b) after 6 hours from loading, (c) after 24 hours from loading, and (d) after 48 hours from loading the inner layer gel.

FIG. 10 shows photograph images showing the state of formulations for a product loaded with two formulations, in which the inner layer gel of prescription 10 is loaded into the outer layer gel of prescription 1 in the Examples, (a) immediately after loading, (b) after 6 hours from loading, (c) after 24 hours from loading, and (d) after 48 hours from loading the inner layer gel.

FIG. 11 shows photograph images showing the state of formulations for a product loaded with two formulations, in which the inner layer gel of prescription 11 is loaded into the outer layer gel of prescription 1 in the Examples, (a) immediately after loading, (b) after 6 hours from loading, (c) after 24 hours from loading, and (d) after 48 hours from loading the inner layer gel.

FIG. 12 shows photograph images showing the state of formulations for a product loaded with two formulations, in which the inner layer gel of prescription 12 is loaded into the outer layer gel of prescription 1 in the Examples, (a) immediately after loading, (b) after 6 hours from loading, (c) after 24 hours from loading, and (d) after 48 hours from loading the inner layer gel.

FIG. 13 shows photograph images showing the state of formulations for a product loaded with two formulations, in which the inner layer gel of prescription 13 is loaded into the outer layer gel of prescription 1 in the Examples, (a) immediately after loading, (b) after 6 hours from loading, (c) after 24 hours from loading, and (d) after 48 hours from loading the inner layer gel.

FIG. 14 shows photograph images showing the state of formulations for a product loaded with two formulations, in which the inner layer gel of prescription 14 is loaded into the outer layer gel of prescription 1 in the Examples, (a) immediately after loading, (b) after 6 hours from loading, (c) after 24 hours from loading, and (d) after 48 hours from loading the inner layer gel.

FIG. 15 shows photograph images showing the state of formulations for a product loaded with two formulations, in which the inner layer gel of prescription 15 is loaded into the outer layer gel of prescription 1 in the Examples, (a) immediately after loading, (b) after 6 hours from loading, (c) after 24 hours from loading, and (d) after 48 hours from loading the inner layer gel.

FIG. 16 shows photograph images showing the state of formulations for a product loaded with two formulations, in which the inner layer gel of prescription 16 is loaded into the outer layer gel of prescription 1 in the Examples, (a) immediately after loading, (b) after 6 hours from loading, (c) after 24 hours from loading, and (d) after 48 hours from loading the inner layer gel.

### DESCRIPTION OF EMBODIMENTS

The present invention is explained hereinafter with exemplary Examples while referring to the appended drawings as needed. Throughout the entire specification, a singular expression should be understood as encompassing the concept thereof in the plural form, unless specifically noted otherwise. Further, the terms used herein should be understood as being used in the meaning that is commonly used in the art, unless specifically noted otherwise. Thus, unless defined otherwise, all terminologies and scientific technical terms that are used herein have the same meaning as the general understanding of those skilled in the art to which the present invention pertains. In case of a contradiction, the present specification (including the definitions) takes precedence.

### Definitions

As used herein. “dosage forms are the same” or “same dosage form” refers to multiple formulations prepared using the same type of substrate. Examples of substrates include, but are not limited to, water soluble substrates, fat and oil substrates, emulsion substrates, gel substrates (such as aqueous gel or oily gel) and the like.

As used herein, “colloid” refers to microparticles of a substance dispersed in a liquid, a semi-solid, or a solid, or the dispersed state of the microparticles (hereinafter, referred to as “colloid particles”). Colloid particles dispersed in a liquid are particularly referred to as “colloid dispersion” or “colloid solution”. In a preferred embodiment, the microparticles are metal microparticles. Such a colloid is referred to as a “metal colloid”. Metal colloid is also called a “metal nanocolloid” particularly when the metal microparticles are nano-sized (nanoparticles).

As used herein, “spherical shape” is not limited to a perfectly spherical shape, but also includes slightly deformed spherical shape (e.g., egg shape), spheroid shape (e.g., shape having an oval cross-section), spherical shape with a small protrusion on the surface, and the like. Similarly, “semispherical shape” as used herein is not limited to a perfectly semispherical shape, but also includes a slightly deformed semispherical shape, a semispherical shape with a small protrusion on the surface, and the like.

As used herein, “aqueous component” refers to a component that can be dissolved and/or diluted by water.

As used herein, “oily component” refers to a substance, which is a liquid or solid at normal temperature, is insoluble to water, is viscous, has a lower specific gravity than water, and is flammable.

As used herein, “emulsion” refers to a homogenously mixture of a liquid, semisolid, or solid which do not dissolve into each other as in an aqueous component and an oily component.

### <Cosmetic Products>

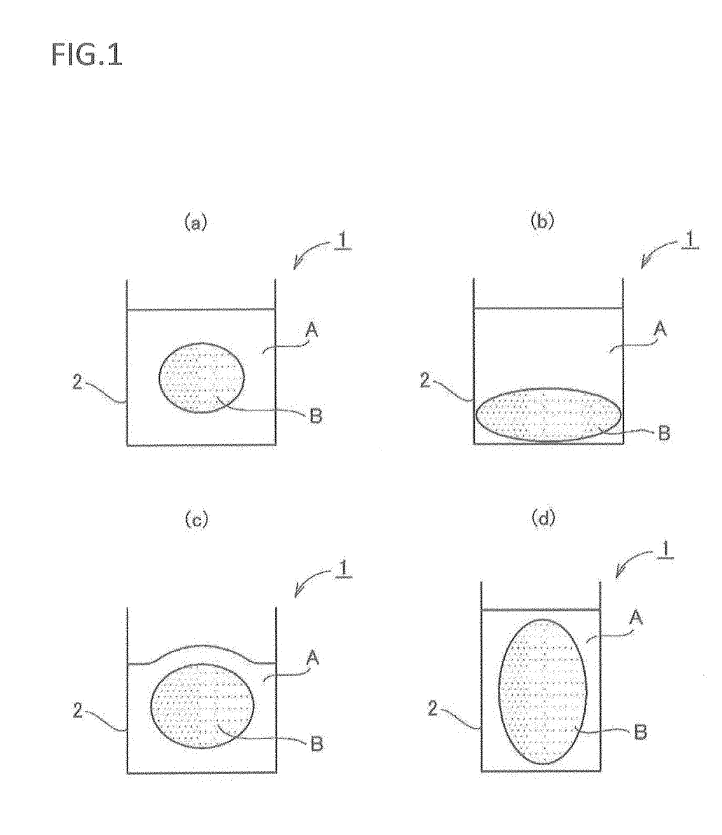

FIGS. 1(a) to (d) are schematic diagrams depicting various forms of a cosmetic product 1 according to one embodiment of the present invention. As shown in FIGS. 1(a) to (d), the cosmetic product 1 has a transparent or semitransparent container 2, a transparent or semitransparent formulation A loaded into the container 2, and a formulation B embedded in the formulation A.

At least a part of the surface of formulation B has a protruding curved surface. For example, as shown in FIG. 1(a), formulation B may be spherical. In such a case, formulation B can be embedded while floating in formulation A. As shown in FIG. 1(b), the bottom portion of formulation B may be in contact with the bottom surface of the container 2. In such a case, the top portion of formulation B would be nearly semispherical. In this regard, formulation B contacts the bottom surface of the container 2 directly or through a thin layer of formulation A. As shown in FIG. 1(c), the top portion near the middle of formulation A may be bulging out while formulation B is surrounded by formulation A. As shown in FIG. 1(d), formulation B may have a long oval shape in the longitudinal direction.

### (1. Container)

As the container 2, a container that allows formulation A and formulation B loaded inside to be viewed from the outside and is used for common cosmetic products such as cream or gel is preferred. The shape, material, and the like thereof are not particularly limited. The shape of the container 2 can be appropriately changed in accordance with the amount of formulation A and formulation B loaded, desired outward appearance, or the like.

### (2. Formulation)

Examples of the dosage form of formulation A and formulation B (hereinafter, also collectively referred to as “formulations”) in this embodiment include, but are not limited to, gel, cream, semisolid, and the like. The color tone of the formulations is also not particularly limited in this embodiment. For example, the formulations may be an opaque, white, transparent, or colored gel or emulsified composition. From the viewpoint of aesthetics of the cosmetic product 1, formulation A in the outer layer is desirably transparent or semitransparent with color or no color, especially when the formulation is aqueous gel. Since this allows the difference in color tones of the outer layer and the inner layer to be visually recognized from the outside of the cosmetic product 1 (from the outside of the container 2), the aesthetics of the cosmetic product 1 is further improved. In a preferred embodiment, formulation A and formulation B have the same dosage form.

Examples of a combination of formulation A and formulation B include, but are not limited to, a combination of two types of “aqueous gel” formulations that replenish moisture and moisturizing components to the skin or the like, a combination of two types of “oily gel” formulations that replenish oily components and moisturizing components to the skin or the like, a combination of “aqueous gel” and “oily gel”, a combination of “aqueous gel cleanser” to which an aqueous cleansing component is added and “oily gel cleanser” to which an oily cleansing component is added, a combination of two types of “cream” formulations that replenish oily components and moisturizing components to the skin or the like, and the like. A combination of two types of “aqueous gel” formulations, a combination of two types of “oily gel” formulations, and a combination of “aqueous gel” and “oily gel” are particularly preferable. From the viewpoint of the aesthetics of cosmetic products, formulation B is preferably transparent or semitransparent.

In this embodiment, at least one of formulation A and formulation B comprises a colloid. Examples of types of colloid particles include, but are not particularly limited to, solids that are insoluble to a dispersion medium (e.g., metal, iron (III) hydroxide, and the like).

In a preferred embodiment, the colloid is a metal colloid. Examples of metal colloids include, but are not limited to, platinum, gold, silver, palladium, rhodium, ruthenium, iridium, and combinations thereof, alloys prepared by mixing these metals, and the like. In particular, platinum, gold, silver, and combinations thereof are more preferable. Colloid particles of metal such as platinum, gold, or silver are generally known to have a negative charge. It is understood that the relationship between the charge of colloid particles (positive or negative charge) and properties of cosmetic component or the like added to formulation B and the dosage form of formulation B can be taken into consideration as needed in selecting the colloid, if necessary. The metal colloid used in this embodiment may be a commercially available product or a metal colloid manufactured by a commonly known manufacturing method.

For example, as a gold colloid, Sigma-Aldrich Co. Ltd.'s Gold colloid, Renaissance Energy Research's nano-gold dispersion, Dainichi Chemical Industry Co., Ltd.'s gold colloid PD-S, or Johzen Co. Ltd.'s gold colloid solution, which are commercially available and prepared as a dispersion, can be directly used. As a silver colloid, Sigma-Aldrich Co, Ltd.'s Silver suspension or Renaissance Energy Research's nano-silver dispersion, which are commercially available and prepared as a dispersion, can be directly used. As a platinum colloid, Renaissance Energy Research's nano-platinum dispersion, which is commercially available and prepared as a dispersion, can be directly used. Johzen Co. Ltd.'s platinum silver colloid 500, which is commercially available and prepared and mixed as a dispersion of platinum colloid and silver colloid, can be directly used.

In one aspect of the present invention, when formulation B is embedded in formulation A, the cosmetic product may be configured such that (1) formulation B comprises a colloid, while formulation A is free of colloid, or (2) formulation A comprises a colloid, while formulation B is free of colloid. In case of (1), the color tone of formulation B in the inner layer can be made darker than the color tone of formulation A in the outer layer so that the contrast in the color tones of the outer layer and the inner layer can be visually recognized from the outside of the container 2. Thus, (1) is more preferable from the viewpoint of the aesthetics of the cosmetic product 1. In case of (2), the difference in color tones of the outer layer and the inner layer from outside of the container 2 may be difficult to visually recognize in a relatively dark room or the like, but the state of multiple layers can be visually recognized when used in a bright room/location, so that the superb aesthetics of the cosmetic product 1 can be observed. Alternatively in another aspect of the present invention, formulation A and formulation B may comprise a colloid at different colloid concentrations. If the concentration of the colloid dispersion added to formulation B in the inner layer is higher in such a case, the color tone of the inner layer can be made to emerge against the outer layer when observing the cosmetic product 1 from the outside of the container 2. Thus, this is more preferable from the viewpoint of aesthetics of the cosmetic product 1. The types of colloid dispersions added to formulation A and formulation B can also be varied to change the color tones of the outer layer and the inner layer to enhance the aesthetics of the cosmetic product 1.

In a preferred aspect of the present invention, formulation B comprises a colloid. The amount of colloid added to formulation B that is embedded in formulation A can be appropriately adjusted in accordance with the combination of formulation A and formulation B, desired color tone of formulation B, or the like. For example, the amount of colloid dispersion added relative to the entire amount of formulation B is 0.001 to 50.00% by weight, preferably 0.005 to 45.00% by weight, and more preferably 0.01 to 43.00% by weight. If the colloid is a metal colloid, the amount of metal colloid dispersion added relative to the entire amount of formulation B is for example 0.5 to 50.00% by weight, preferably 1.0 to 45.00% by weight, and more preferably 2.0 to 43.00% by weight.

The average particle size of colloid particles is not particularly limited. For example, the average particle size is 1 nm to 10 μm, preferably 1 nm to 7 μm, and more preferably 1 nm to 5 μm. If the colloid is a metal colloid, the average particle size of the metal colloid is not particularly limited, but a 1 nm to 100 nm metal colloid is preferably used and a 10 nm to 100 nm metal colloid is more preferably used in terms of the stability of metal colloid in formulation B.

The average particle size of colloid particles is measured in accordance with a known method, such as dynamic light scattering or laser diffraction. For example, if nano-sized colloid particles of metal colloid or the like are used, it is preferable to measure the average particle size using dynamic light scattering. The dispersiveness of colloid particles can also be evaluated by measuring the zeta potential together with the average particle size.

Metal colloids, especially metal nano-colloids, are known to exhibit bright color tones when the optical electric field of the visible to far infrared regions couples with plasmons, resulting in light absorption. In this embodiment, the aesthetics of the cosmetic product 1 can thus be adjusted by adjusting the type or amount of metal colloid dispersion added to formulation A and/or formulation B, the average particle size of metal colloid particles, or the like. For example, when highly transparent aqueous gel that does not contain a colloid is used as formulation A in the outer layer and a metal colloid dispersion is added to formulation B in the inner layer, the coloration of the metal colloid contained in formulation B in the inner layer can be seen through formulation A from the outside of the container 2, so that cosmetic product 1 with super aesthetic can be prepared with the contrast in the color tones between the outer layer and the inner layer. When a metal colloid dispersion is added to both formulation A and formulation B, the color tone of the inner layer can be made to emerge against the outer layer by increasing the concentration of metal colloid dispersion added to formulation B in the inner layer to enhance the aesthetics of the cosmetic product 1,

The types of metal colloid dispersions added between formulation A and formulation B can also be varied to change the color tones of the outer layer and the inner layer and enhance the aesthetics of the cosmetic product 1. When highly transparent colored aqueous gel with any colorant added thereto is used as formulation A and/or formulation B, the aesthetics of the cosmetic product 1 can be enhanced by the additive or synergistic effect with the color tone of the metal colloid contained in formulation A and/or formulation B.

While the viscosity of formulation A and formulation B is not particularly limited in this embodiment, if the viscosity is too low, formulation B moves to or mixes with formulation A on the outside due to an impact or the like, such that the shape after loading cannot be stably maintained in some cases. If the viscosity is too high, it can be difficult to load a formulation or such high viscosity can impair the usability of a product.

It should be noted that a bilayer state, where formulation B is included inside formulation A, is not maintained in this embodiment by providing a viscosity gradient in formulation A in the outer layer and formulation B in the inner layer. In other words, it is not necessary in this embodiment to intentionally provide a difference in viscosity between formulation A and formulation B.

In any case, a formulation that does not have fluidity and is not too hard is desirable as formulation A and formulation B. Furthermore, formulation A in the outer layer and formulation B in the inner layer are desirably formulations with similar levels of viscosity in view of the ease of use of the formulations, ease of loading in the production process, need for loading formulation B in the inner layer in an approximately spherical shape, and the like.

The viscosity of a formulation can be represented by the maximum load according to the following measurement method. The maximum load of a formulation is preferably 0.2 N to 2.0 N and more preferably 0.3 N to 1.0 N. If the maximum load is less than 0.2 N, it can be difficult to maintain a stable shape of a formulation as discussed above. If the maximum load is greater than 2.0 N, it can be difficult to load a formulation or such a high viscosity can impair the usability of a product.

The maximum load of a formulation is measured with SUN RHEO METER (COMPAC 100-II) purchased from Sun Scientific Co., Ltd., which measures the maximum value of load applied per cross-sectional area of a rod-like probe with a diameter of 20 mm when the rod-like probe is inserted 25 mm at a rate of 60 mm/min. The maximum load is preferably measured at room temperature, such as 20° C., 25° C., or 30° C. and typically at 25° C.

While the specific gravity of each formulation can be adjusted depending on the application and prescription of each formulation, the specific gravity of formulation A is preferably 0.8 to 1.3 and more preferably 0.9 to 1.2. The specific gravity of formulation B is preferably 0.7 to 1.2 and more preferably 0.8 to 1.1.

The ratio of formulation A and formulation B in the cosmetic product 1 can be adjusted to any value depending on the application and prescription of each formulation. In view of the aesthetics of the cosmetic product 1, the ratio of formulation B relative to the entire amount of the formulations is preferably 10 to 60% by weight and more preferably 20 to 40% by weight. If the ratio of formulation B is less than 10% by weight of the entire amount of formulations, the function of formulation B may not be exerted. If the ratio exceeds 60% by weight, the function of formulation A may not be exerted and the outward appearance of the cosmetic product 1 may be impaired.

In this embodiment, any water soluble substance or any substance or solid that can be dispersed in gel can be added to formulation A and formulation B. Examples of components that can be added to a formulation include cosmetic product substrates such as fats and oils, waxes, hydrocarbons, silicones, fatty acids, alcohols, esters, surfactants, thickeners, and powders, as well as pharmaceuticals, active ingredients of quasi drugs, pH adjusting agents, preservatives, pigments, fragrance, antioxidants, and naturally derived extracts.

In this embodiment, an oily component may be added to formulation A and formulation B. For example, such an oily component can be, but is not limited to, a substance selected from the group consisting of the following: fats and oils such as avocado oil, almond oil, olive oil, camellia oil, sesame oil, rice bran oil, safflower oil, soybean oil, corn oil, rapeseed oil, apricot kernel oil, persic oil, peach kernel oil, castor oil, sunflower oil, grape seed oil, cottonseed oil, coconut oil, wheat germ oil, rice germ oil, evening primrose oil, hybrid sunflower oil, macadamia nut oil, meadowfoam oil, hazelnut oil, palm kernel oil, palm oil, coconut oil, cocoa butter, shea butter, wood wax, mink oil, turtle oil, egg yolk oil, beef tallow, milk fat, lard, and horse oil; waxes such as jojoba oil, carnauba wax, candelilla wax, rice bran wax, orange roughy oil, beeswax, shellac, lanolin, and montan wax; hydrocarbons such as squalene, squalane, liquid paraffin, paraffin, microcrystalline wax, Vaseline, soft fluid isoparaffin, hydrogenated polyisobutylene, ozocerite, ceresin, α-olefin oligomer, polybutene, and polyethylene; higher fatty acids such as lauric acid, myristic acid, palmitic acid, stearic acid, behenic acid, hydroxystearic acid, oleic acid, linoleic acid, ethylhexanoic acid, isostearic acid, isopalmitic acid, isotridecanoic acid, isononanoic acid, and pentadecanoic acid; higher alcohols such as lauryl alcohol, stearyl alcohol, cetearyl alcohol, behenyl alcohol, cetanol, oleyl alcohol, lanolin alcohol, cholesterol, isocholesterol, sitosterol, stigmasterol, isostearyl alcohol, octyldodecanol, and hexyldecanol; esters such as isopropyl myristate, isopropyl palmitate, butyl stearate, ethyl oleate, cetyl palmitate, myristyl myristate, octyldodecyl myristate, octyldodecyl oleate, cholesteryl stearate, cholesteryl hydroxystearate, tricaprin, trimyristin, glyceryl tri-2-ethylhexanoate (trioctanoin), isopropyl isostearate, isopropyl isostearate, ethyl isostearate, cetyl ethylhexanoate, stearyl ethylhexanoate, glyceryl triethylhexanoate, glyceryl triethylhexanoate, trimethylolpropane triethylhexanoate, pentaerythril tetraethylhexanoate, glyceryl triisostearate, trimethylolpropane triisostearate, pentaerythril tetraisostearate, pentaerythril triisostearate, isocetyl isostearate, octyldodecyl dimethyl octanoate, myristyl lactate, cetyl lactate, trioctyldodecyl citrate, and diisostearyl malate; and a mixture of two of more thereof.

In this embodiment, a moisturizing component may be added to formulation A and formulation B. For example, such a moisturizing component can be, but is not limited to, a substance selected from the group consisting of the following:

glycerin, 1,3-butylene glycol, propylene glycol, 3-methyl-1,3-butanediol, 1,3-propanediol, 2-methyl-1,3-propanediol, trimethylolpropane, pentaerythritol, hexylene glycol, diglycerin, polyglycerin, diethylene glycol, polyethylene glycol, dipropylene glycol, polypropylene glycol, ethylene glycol, diethylene glycol monoethyl ether (ethoxydiglycol), ethylene glycol monoethyl ether, ethylene glycol monobutyl ether, diethylene glycol dibutyl ether, sorbitol, xylitol, erythritol, mannitol, maltitol, glucose, fructose, galactose, mannose, threose, xylose, arabinose, fucose, ribose, deoxyribose, maltose, trehalose, lactose, raffinose, gluconic acid, glucuronic acid, cyclodextrin, β-glucan, chitin, chitosan, heparin and derivatives thereof, pectin, arabinogalactan, dextrin, dextran, glycogen, ethyl glucoside, glucosyl ethyl methacrylate polymer, hyaluronic acid, sodium hyaluronate, mucoitin sulfuric acid, charonin sulfate, keratosulfate, dermatan sulfate, Tremella fuciformis extract, Tremella fuciformis polysaccharide, tuberose polysaccharide, citric acid, tartaric acid, urea, salts of 2-pyrrolidone-5-carboxylic acid and sodium thereof, betaine (trimethylglycine), proline, hydroxyproline, arginine, lysine, serine, glycine, alanine, phenylalanine, tyrosine, β-alanine, threonine, glutamic acid, glutamine, asparagine, aspartic acid, cysteine, cystine, methionine, leucine, isoleucine, valine, tryptophan, histidine, taurine, collagen, atelocollagen, gelatin, elastin, peptide from decomposed collagen, hydrolyzed collagen, hydroxypropylammonium chloride hydrolyzed collagen, peptide from decomposed elastin, peptide from decomposed keratin, hydrolyzed keratin, peptide from decomposed conchiolin, hydrolyzed conchiolin, peptide from decomposed silk protein, hydrolyzed silk, sodium lauroyl hydrolyzed silk, peptide from decomposed soy protein, peptide from decomposed wheat protein, hydrolyzed wheat protein, peptide derived from decomposed casein, acylated peptide, other protein peptides and derivatives thereof; palmitoyl oligopeptide, palmitoyl pentapeptide, palmitoyl tetrapeptide, lactic acid bacteria culture, yeast extract, eggshell membrane protein, bovine submaxillary mucin, hypotaurine, sesame lignan glycoside, glutathione, albumin, and whey; choline chloride and phosphorylcholine; and placenta extract, elastin, collagen, aloe extract, Hammamelis virginiana water, Luffa cylindrica water, Chamomilla recutita extract, licorice extract, comfrey extract, silk extract, Rosa roxburghii extract, Achillea millefolium extract, Eucalyptus globulus extract, melilot extract, natural ceramides (types 1, 2, 3, 4, 5, and 6), hydroxyceramide, and glycosphingolipid.

In this embodiment, various cosmetic components that can be commonly added to cosmetic products can also be added to formulation A and formulation B in addition to or instead of the above components. In particular, when a cosmetic component that is a macromolecular compound is added to formulation B in the inner layer, inclusion of a colloid in formulation B can inhibit the dispersion of the cosmetic component from the inner layer to the outer layer at the interface between the outer layer and the inner layer, so that the cosmetic component can be retained in the inner layer for a long period of time. For this reason, multiple formulations can be used separately more efficiently with one product.

A dye may be appropriately added to formulation A and/or formulation B of the present invention. A dye to be added is not particularly limited, as long as it is a dye that disperses homogeneously in a formulation. Representative dyes include, but are not limited to, Red No. 2, Red No. 3, Red No. 102, Red No. 106, Yellow No. 4, Yellow No. 5, Green No. 3, Blue No. 1, Blue No. 2, Red No. 201, Red No. 213, Red No. 214, Red No. 227, Red No. 231, Red No. 232, Orange No. 205, Orange No. 207, Yellow No. 203, Green No. 201, Green No. 204, Green No. 205, Blue No. 202, Blue No. 203, Blue No. 205, Blue No. 205, Brown No. 201, Red No. 401, Red No. 502, Red No. 503, Red No. 504, Red No. 506, Orange No. 402, Yellow No. 402, Yellow No. 406, Yellow No. 407, Green No. 401, Green No. 402, Purple No. 401, Black No. 401, and other artificial coloring, gardenia dye, cyanocobalamin, Lithospermum erythrorhizon extract, axulene and derivatives thereof, chlorophyll and derivatives thereof, cochineal dye, and other dyes and components with a coloring property. These dyes can be added alone or in any combination.

Since at least one of formulation A and formulation B comprises a colloid or they comprise different types of colloids in the cosmetic product 1 according to this embodiment, mixture of formulation A and formulation B at the interface is inhibited, so that a multilayer state where formulation B is included inside formulation A can be stably retained for a longer period of time. For this reason, the cosmetic product 1 according to this embodiment can maintain a multilayer state not only when the cosmetic product is unused (before breaking the seal), but also when the amount of the formulations decreases from use after breaking the seal of the cosmetic product 1. Therefore, the cosmetic product 1 according to this embodiment not only has superb ability to retain the shape of formulations, but also is capable of stably maintaining the aesthetics for a longer period of time. An effect of improving impression upon use for a user (actual feeling of applying a cosmetic component of interest to the skin or the like) and the like is also expected.

While this embodiment explained a case where two types of formulations (formulation A and formulation B) are used, the effect of the present invention can also attained when two or more formulations are used as the cosmetic product of the present invention. In other words, a formulation with the same dosage form as formulations A and B can be included inside formulation B to prepare a cosmetic product with a structure of three or more layers. It is also possible to embed formulation B in the inner layer at multiple locations inside formulation A in the outer layer (formation B in the inner layer exhibiting an outward appearance of polka dot patterns).

### <Method of Manufacturing Cosmetic Products>

The method of manufacturing cosmetic products of the present invention is explained in this section. The cosmetic product 1 according to this embodiment can be manufactured by first loading the container 2 with formulation A in the outer layer, and then loading formulation B in the inner layer inside formulation A. The method of loading formulation A and formulation B is not particularly limited. Each of the formulations can be loaded using a loading machine, an injector, or the like that is commonly used in a manufacturing process of cosmetic products while being rotated or directly.

For example, a cosmetic product can be manufactured by first inserting a nozzle inside pre-loaded formulation A and loading formulation B. Depending on the combination of formulation A and formulation B, a protrusion may be formed on top of the surface forming a protruding curved surface of formulation B when withdrawing the nozzle after loading formulation B, but this would not impair the aesthetics of the cosmetic product 1 or have any effect on the completion of the invention.

In this embodiment, cosmetic product 1 having the functionalities of two formulations, formulation A and formulation B, can be provided in a single product by loading two types of formulations with different functions in two phases. This embodiment can also provide, for example, a novel cosmetic product, which uses formulation A such as aqueous gel with high transparency in the outer layer and has formulation B comprising a colloid loaded inside formulation A so that formulation B having a curved surface and a color tone exhibited by the colloid can be visually recognized from the outside through the container 2 and formulation A, thus having superb aesthetics.

The method of preparing formulation A and formulation B is not particularly limited. The formulations can be prepared in accordance with a known method. For example, if formulation A and formulation B are aqueous gel, a desired cosmetic component or the like can be measured out to a predetermined ratio, and heated and dissolved while stirring, and then any pH adjusting agent (e.g., potassium hydroxide, sodium hydroxide, or the like) dissolved at any ratio using purified water is added, then the mixture is cooled to room temperature while stirring to prepare the aqueous gel. The method of adding a colloid to formulation A and/or formulation B is also not particularly limited. For example, if a formulation is aqueous gel, a colloid can be added together when the cosmetic component is heated and dissolved while being stirred, or a colloid may be added under heating conditions after adding the pH adjusting agent.

In this embodiment, the ratio of the amount of formulation A first loaded into the container 2 and the amount of formulation B loaded in the second phase is not particularly limited. The ratio is appropriately adjusted depending on the type or functionality of the selected formulation, properties required in the cosmetic product 1 or the like.

From the viewpoint of aesthetics of the cosmetic product 1 in this embodiment, the amount of formulation B in the inner layer which is loaded in the second phase is desirably within the range of 10% to 60% and more desirably within the range of 20 to 40% of the entire formulations, but the ratio is not limited thereto. If the amount of formulation B is less than 10% of the entire formulations, the function of formulation B may not be exerted. If the amount exceeds 60%, the function of formulation A may not be exerted and the outward appearance of the cosmetic product 1 may be impaired.

If the container 2 is a shallow container, a formation loaded in the second phase or thereafter contacts the bottom surface of the container 2 and is loaded in a semispherical or semi-spheroid shape depending on the amount of the formulation loaded in the second phase or thereafter, but this would not impair the functionality of the present invention, or impair the aesthetics of the invented product, or have any effect on the practice of the invention. If the depth of the container 2 is sufficiently deep or depending on the shape of the container 2, the amount of the formulation loaded in the second phase can be adjusted to be loaded in a completely spherical shape. This enables the final invented product to have a slightly different outward appearance from the former.

Formulation B loaded in the second phase needs to be loaded inside formulation A that has been first loaded, but the location to where formation B is loaded is not particularly limited. It is generally desirable to load formulation B near the center of formulation A that has been loaded first, but formulation B may be loaded at a position above or below the center of formulation A that has been loaded first depending on the hardness, shape, or functionality of a formulation, or the ratio at which the formulation is added. By loading formulation B above the center, formation B is less likely to adhere to the bottom portion of the container 2, and can be loaded in a shape closer to a sphere, whereby the top portion of formulation A often bulges out.

The cosmetic product 1 obtained in this manner can have the functionalities of both formulation A in the outer layer and formulation B in the inner layer and stably retain a multilayer state having formulation B included inside formulation inside formulation A for long period of time. Further, the cosmetic product 1 can be prepared to have superb aesthetics when formulation A and formulation B are viewed through the container 2 from both the overall unity from combining formulation A and formulation B and the difference in color tones (contrast) between formulations due to formulation A and/or formulation B comprising a colloid.

### OTHER EMBODIMENTS

The present invention has been explained while showing preferred embodiments to facilitate understanding. While the present invention is explained hereinafter based on Examples, the above explanation and the following Examples are not provided to limit the present invention, but for the sole purpose of exemplification. It is understood that those skilled in the art can implement an equivalent scope from the descriptions of the specific preferred embodiments of the invention based on the description of the present invention and common general knowledge. The scope of the present invention is therefore not limited to the embodiments and Examples that are specifically described herein and is limited only by the scope of claims.

### EXAMPLES

### <Preparation of Formulation in the Outer Layer>

A formulation in the outer layer was prepared by the following prescription and manufacturing method.

### (Prescription 1: Formulation in the Outer Layer/Transparent Aqueous Gel)

(A) Acrylic acid/alkylmethacrylic acid polymer: 0.7 (% by weight)
(B) Potassium hydroxide: 0.26 (% by weight)
(A) Concentrated glycerin: 15 (% by weight)
(A) 1,3-butylene glycol: 5 (% by weight)
(A) Methyl parahydroxybenzoate: 0.15 (% by weight)
(A) Purified water: added as the remainder so that the total amount would be 100 (% by weight)

### (Preparation Method)

After heating and dissolving component (A) while stirring, component (B) dissolved be to 10% using purified water was added. The mixture was then cooled to room temperature while stirring, resulting in transparent gel.

### (Measurement of Physical Property Values)

After preparing the gel formulation of prescription 1, 100 g thereof was placed in a 100 ml beaker and was left standing at room temperature for 24 hours to measure the maximum load and specific gravity. As a result thereof, the maximum load was 0.54 N and the specific gravity was 1.03. The maximum load of the gel formulation was measured with SUN RHEO METER (COMPAC 100-II) purchased from Sun Scientific Co., Ltd. by measuring the maximum value of load applied per cross-sectional area of a rod-like probe with a diameter of 20 mm when the rod-like probe was inserted 25 mm at a rate of 60 mm/min.

### <Preparation of Formulation in the Inner Layer>

A formulation in the inner layer was prepared by the following prescription and manufacturing method.

(Prescriptions 2 to 16: Formulation in Inner Layer/Aqueous Gel with Color)
(A) Acrylic acid/alkylmethacrylic acid polymer: 0.7 (% by weight)
(B) Potassium hydroxide: 0.26 (% by weight)
(A) Concentrated glycerin: 15 (% by weight)
(A) 1,3-butylene glycol: 5 (% by weight)
(A) Methyl parahydroxybenzoate 0.15 (% by weight)
(C) Dye component or colloid (shown in the following Table 1)
(A) Purified water: added as the remainder so that the total amount would be 100 (% by weight)

### (Preparation Method)

After heating and dissolving component (A) while stirring, component (B) dissolved to be 10% using purified water was added. The dye of component (C) was added under the addition conditions shown in Table 1, and the mixture was cooled to room temperature while stirring to obtain colored gel. Johzen Co. Ltd.'s platinum silver colloid 500 was used as the platinum silver colloid of prescription 13, Dainichi Chemical Industry Co., Ltd.'s gold colloid PD-S was used as the gold colloid of prescription 14, and Sigma-Aldrich Co. Ltd.'s Silver suspension was used as the silver colloid of prescription 15.

TABLE 1

| | | Amount | |
|---|---|---|---|
| Prescription | Dye component or | added (% | Addition |
| No. | colloid | by weight) | conditions |
| Prescription 2 | Safflower yellow | 0.05 | Added at 40° C. |
| | dye, ethanol | | after adding (B) |
| Prescription 3 | Gardenia yellow, | 0.04 | Added at 40° C. |
| | fructose, | | after adding (B) |
| | tetrapotassium | | |
| | pyrophosphate | | |
| Prescription 4 | Cochineal, ethanol, | 0.06 | Added at 40° C. |
| | citric acid | | after adding (B) |
| Prescription 5 | Laccaic acid, | 0.04 | Added at 40° C. |
| | glycerin, ethanol | | after adding (B) |
| Prescription 6 | Laccaic acid, | 0.1 | Added at 40° C. |
| | disodium tartrate, | | after adding (B) |
| | sulfuric acid | | |
| | (Al/ammonium), | | |
| | sodium carbonate | | |
| Prescription 7 | Cochineal, disodium | 0.1 | Added at 40° C. |
| | tartrate, sulfuric | | after adding (B) |
| | acid (Al/ammonium), | | |
| | sodium carbonate | | |
| Prescription 8 | Gardenia blue, | 0.01 | Added at 40° C. |
| | dextrin | | after adding (B) |
| Prescription 9 | Yellow No. 4 | 0.005 | Added at 40° C. |
| | | | after adding (B) |
| Prescription 10 | Red No. 401 | 0.0002 | Added at 40° C. |
| | | | after adding (B) |
| Prescription 11 | Green No. 204 | 0.002 | Added at 40° C. |
| | | | after adding (B) |
| Prescription 12 | Black No. 401 | 0.0006 | Added at 40° C. |
| | | | after adding (B) |
| Prescription 13 | Platinum silver | 7 | Added at 40° C. |
| | colloid | | after adding (B) |
| Prescription 14 | Gold colloid | 2 | Added at 40° C. |
| | | | after adding (B) |
| Prescription 15 | Silver colloid | 42.25 | Added with (A) |
| | | | while stirring |
| Prescription 16 | India ink | 0.001 | Added at 40° C. |
| | | | after adding (B) |

### <Loading a Formulation>

After loading 20 g of outer layer gel of prescription 1 in a transparent resin container with a 30 g capacity with a syringe, the tip of the syringe was inserted into the loaded outer layer gel to load 10 g each of inner layer gel

### (Prescriptions 2 to 16).

### <Evaluation of Retention of the Shape of Formulation>

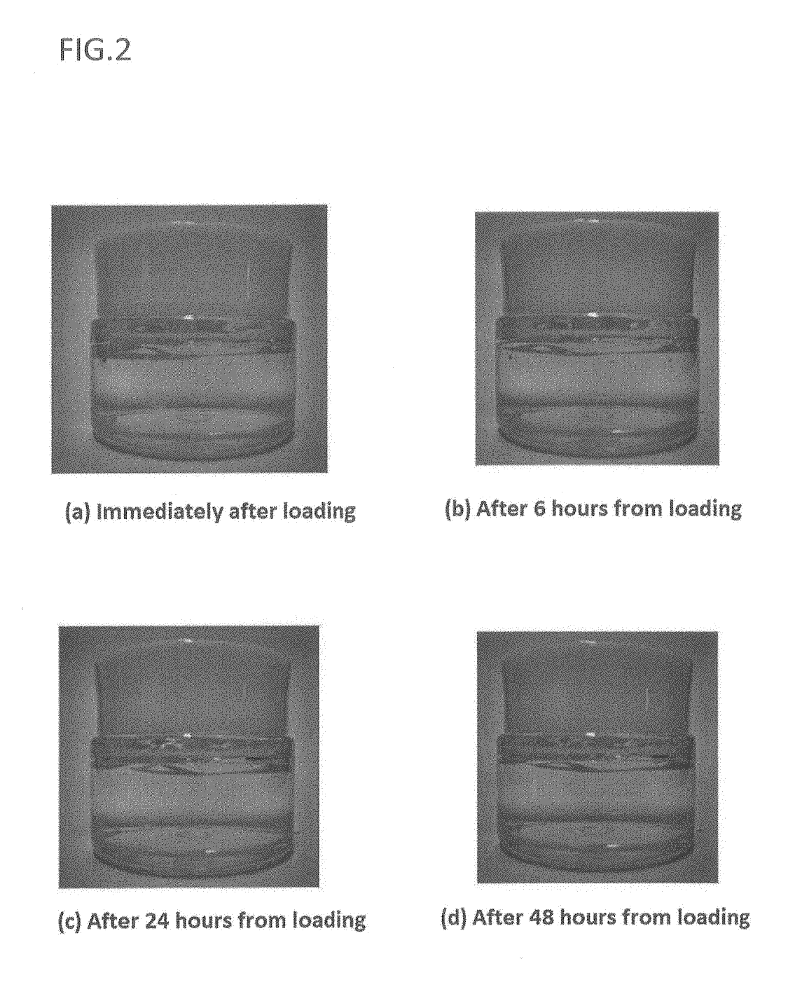

A product prepared by loading two types of different gel formulations (product loaded with two formulations) by the above method was stored at room temperature. The formulations were visually observed immediately after loading, after 6 hours from loading, after 24 hours from loading, and after 48 hours from loading the inner layer gel. The results are shown in FIGS. 2 to 16.

FIG. 2 shows photograph images showing the state of formulations for a product loaded with two formulations, in which the inner layer gel of prescription 2 is loaded into the outer layer gel of prescription 1 in the Examples, (a) immediately after loading, (b) after 6 hours from loading, (c) after 24 hours from loading, and (d) after 48 hours from loading the inner layer gel.

As can be understood from FIG. 2, in this product, the semispherical inner layer gel was included inside the outer layer gel immediately after loading the bright yellow inner layer gel, but the boundary between the inner layer gel and the outer layer gel became unclear after 6 hours from loading. The semispherical shape of the inner gel could not be observed after 24 hours from loading.

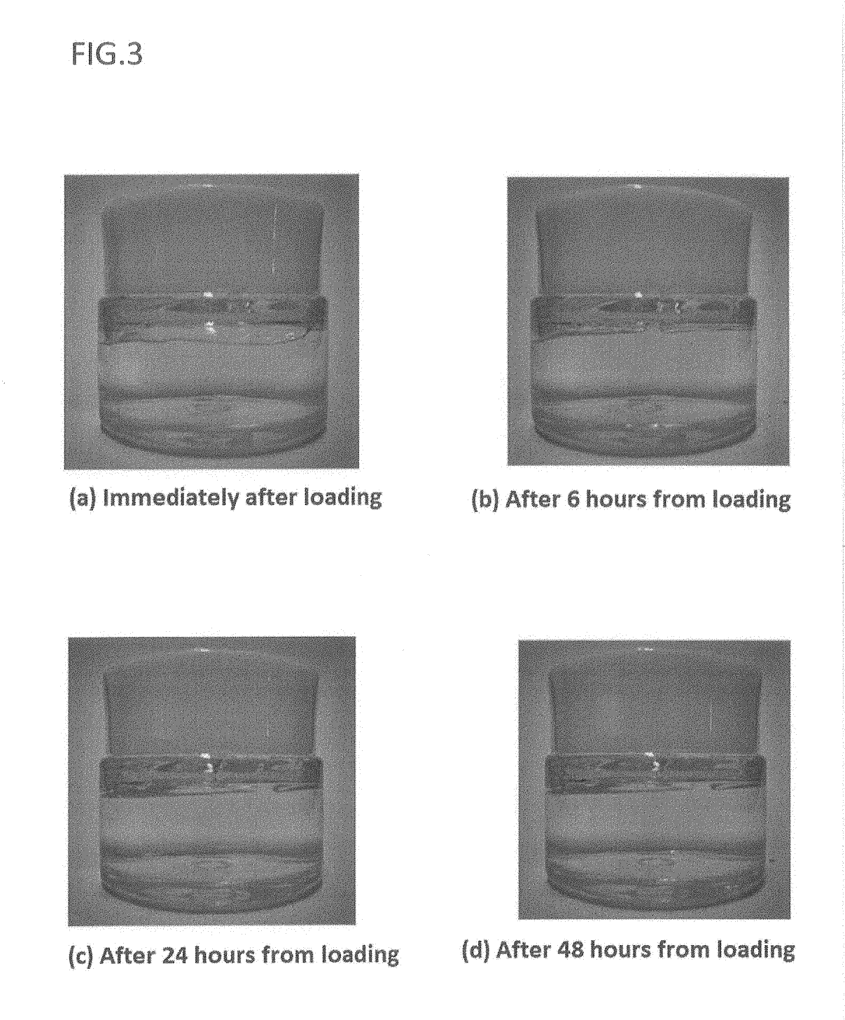

FIG. 3 shows photograph images showing the state of formulations for a product loaded with two formulations, in which the inner layer gel of prescription 3 is loaded in the outer layer gel of prescription 1, (a) immediately after loading, (b) after 6 hours from loading, (c) after 24 hours from loading, and (d) after 48 hours from loading the inner layer gel.

As can be understood from FIG. 3, in this product, the semispherical inner layer gel was included inside the outer layer gel immediately after loading the bright yellow inner layer gel, but the boundary between the inner layer gel and the outer layer gel became unclear after 6 hours from loading. The semispherical shape of the inner gel could not be observed after 24 hours from loading.

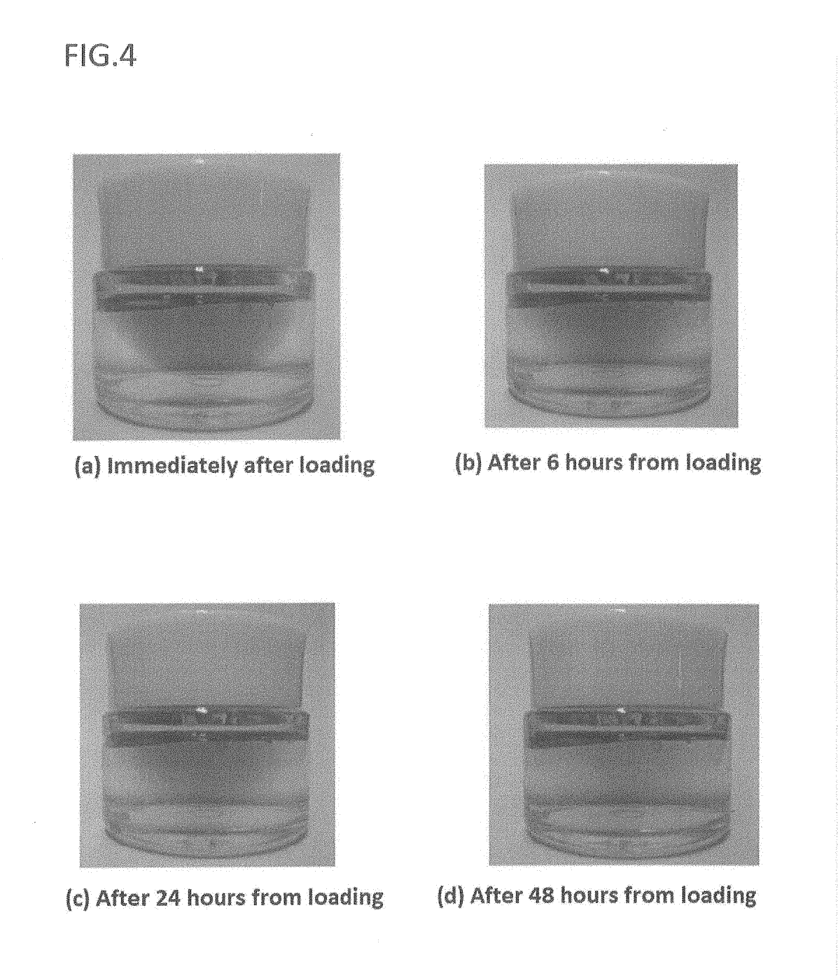

FIG. 4 shows photograph images showing the state of formulations for a product loaded with two formulations, in which the inner layer gel of prescription 4 is loaded into the outer layer gel of prescription 1, (a) immediately after loading, (b) after 6 hours from loading, (c) after 24 hours from loading, and (d) after 48 hours from loading the inner layer gel.

As can be understood from FIG. 4, in this product, the semispherical inner layer gel was included inside the outer layer gel immediately after loading the bright purple inner layer gel, but the boundary between the inner layer gel and the outer layer gel became unclear after 6 hours from loading. The semispherical shape of the inner gel could not be observed after 24 hours from loading.

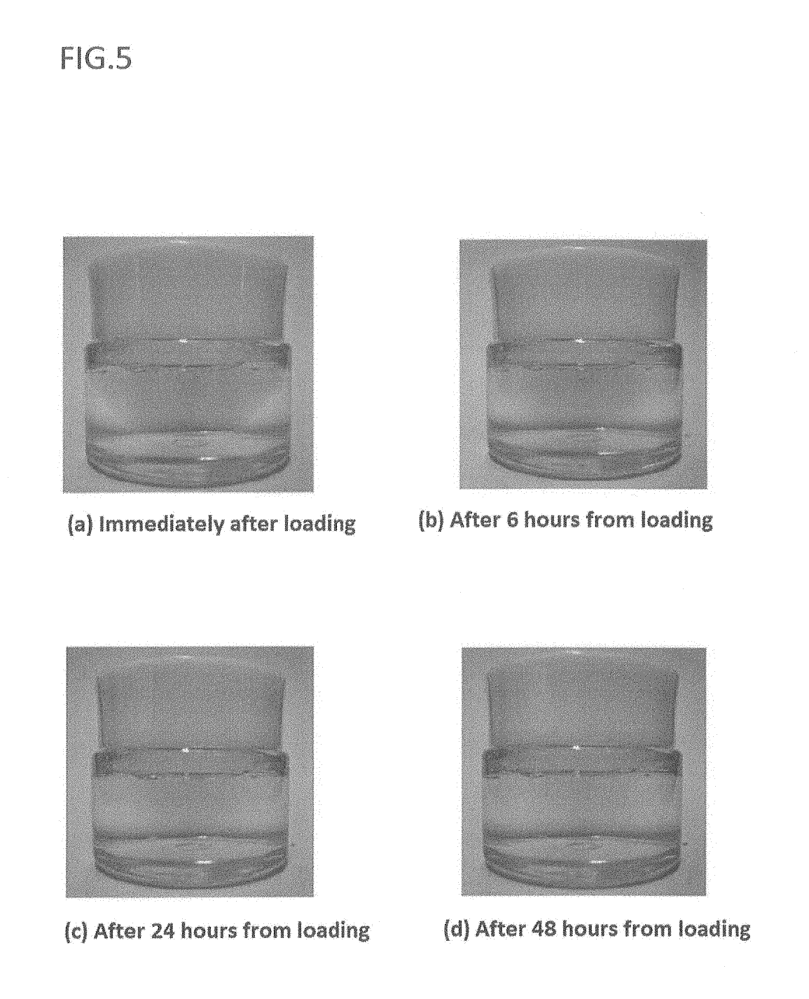

FIG. 5 shows photograph images showing the state of formulations for a product loaded with two formulations, in which the inner layer gel of prescription 5 is loaded into the outer layer gel of prescription 1, (a) immediately after loading, (b) after 6 hours from loading, (c) after 24 hours from loading, and (d) after 48 hours from loading the inner layer gel.

As can be understood from FIG. 5, in this product, the semispherical inner layer gel was included inside the outer layer gel immediately after loading the bright purple inner layer gel, but the boundary between the inner layer gel and the outer layer gel became unclear after 6 hours from loading. The semispherical shape of the inner gel could not be observed after 24 hours from loading.

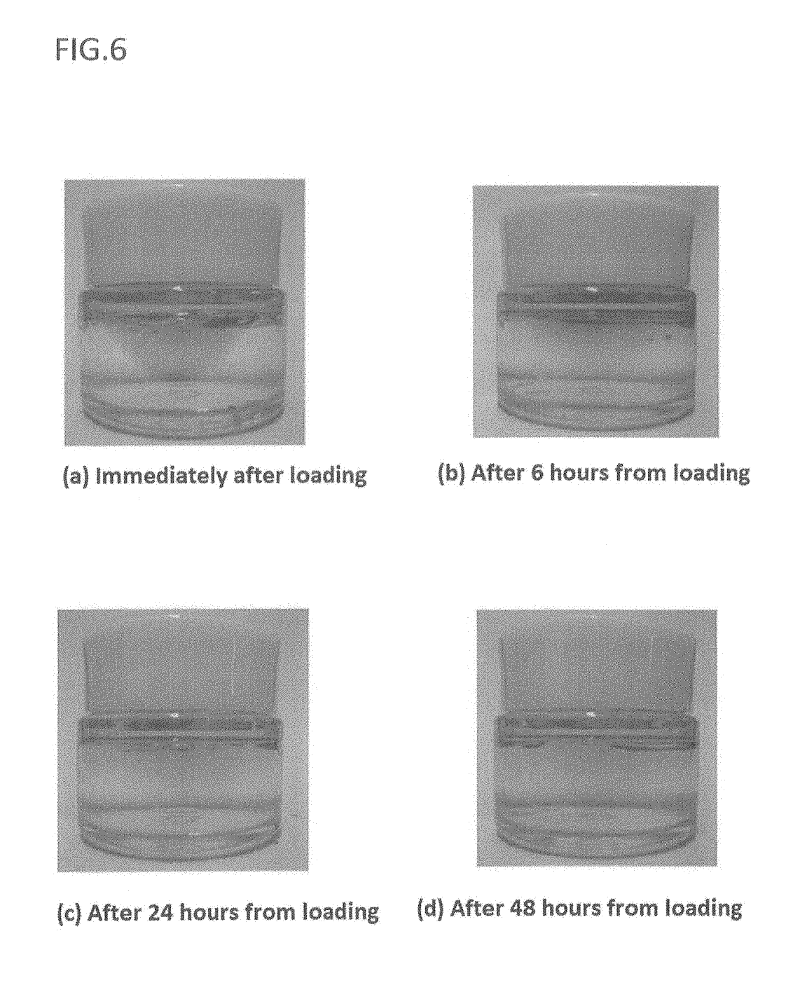

FIG. 6 shows photograph images showing the state of formulations for a product loaded with two formulations, in which the inner layer gel of prescription 6 is loaded into the outer layer gel of prescription 1, (a) immediately after loading, (b) after 6 hours from loading, (c) after 24 hours from loading, and (d) after 48 hours from loading the inner layer gel.

As can be understood from FIG. 6, in this product, the semispherical inner layer gel was included inside the outer layer gel immediately after loading the bright purple inner layer gel, but the boundary between the inner layer gel and the outer layer gel became unclear after 6 hours from loading. The semispherical shape of the inner gel could not be observed after 24 hours from loading.

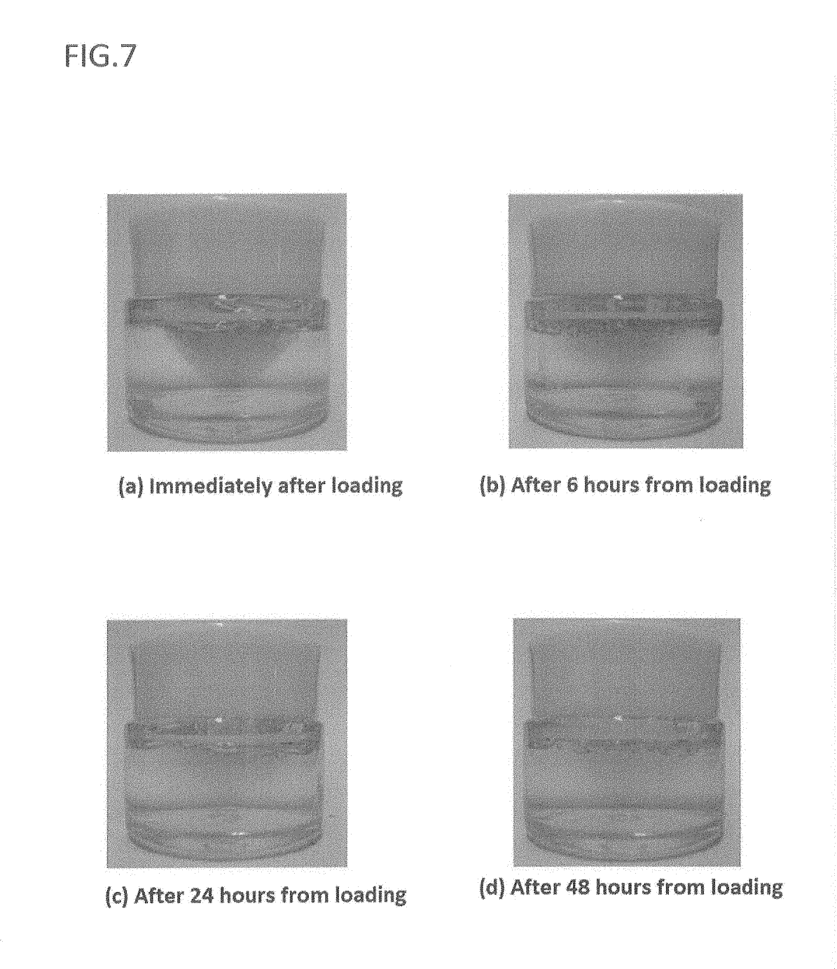

FIG. 7 shows photograph images showing the state of formulations for a product loaded with two formulations, in which the inner layer gel of prescription 7 is loaded into the outer layer gel of prescription 1, (a) immediately after loading, (b) after 6 hours from loading, (c) after 24 hours from loading, and (d) after 48 hours from loading the inner layer gel.

As can be understood from FIG. 7, in this product, the semispherical inner layer gel was included inside the outer layer gel immediately after loading the bright purple inner layer gel, but the boundary between the inner layer gel and the outer layer gel became unclear after 6 hours from loading. The semispherical shape of the inner gel could not be observed after 24 hours from loading.

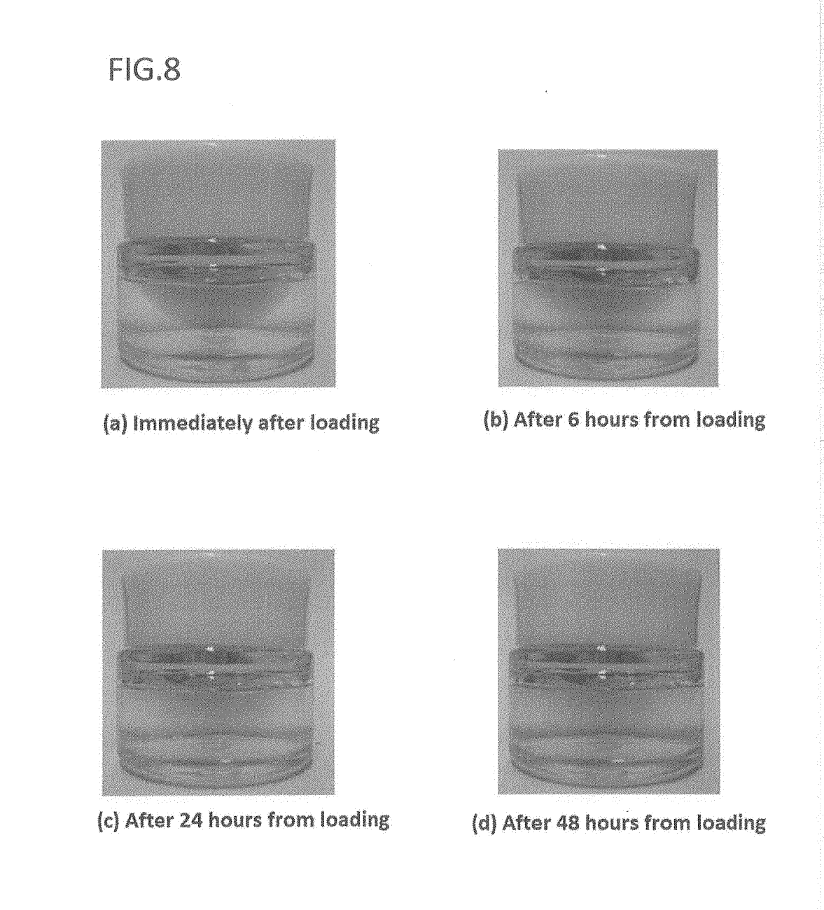

FIG. 8 shows photograph images showing the state of formulations for a product loaded with two formulations, in which the inner layer gel of prescription 8 is loaded into the outer layer gel of prescription 1, (a) immediately after loading, (b) after 6 hours from loading, (c) after 24 hours from loading, and (d) after 48 hours from loading the inner layer gel.

As can be understood from FIG. 8, in this product, the semispherical inner layer gel was included inside the outer layer gel immediately after loading the bright blue inner layer gel, but the boundary between the inner layer gel and the outer layer gel became unclear after 6 hours from loading. The semispherical shape of the inner gel could not be observed after 24 hours from loading.

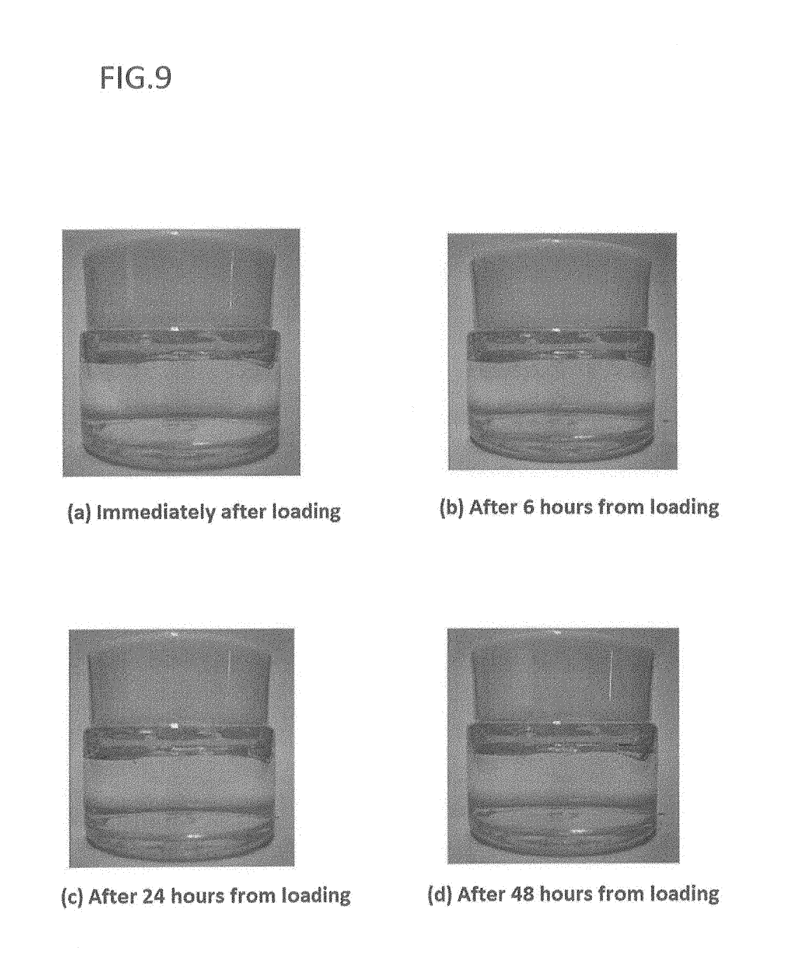

FIG. 9 shows photograph images showing the state of formulations for a product loaded with two formulations, in which the inner layer gel of prescription 9 is loaded into the outer layer gel of prescription 1, (a) immediately after loading, (b) after 6 hours from loading, (c) after 24 hours from loading, and (d) after 48 hours from loading the inner layer gel.

As can be understood from FIG. 9, in this product, the semispherical inner layer gel was included inside the outer layer gel immediately after loading the bright yellow inner layer gel, but the boundary between the inner layer gel and the outer layer gel became unclear after 6 hours from loading. The inner layer gel and the outer layer gel were homogenously mixed after 24 hours from loading.

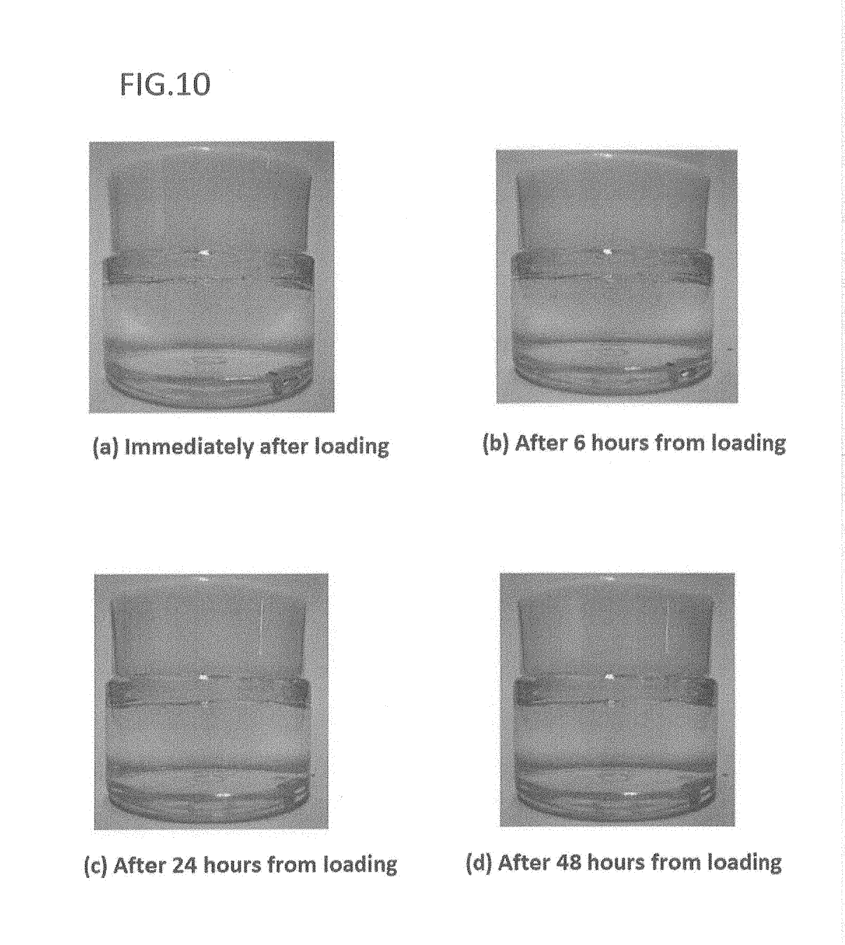

FIG. 10 shows photograph images showing the state of formulations for a product loaded with two formulations, in which the inner layer gel of prescription 10 is loaded into the outer layer gel of prescription 1, (a) immediately after loading, (b) after 6 hours from loading, (c) after 24 hours from loading, and (d) after 48 hours from loading the inner layer gel.

As can be understood from FIG. 10, in this product, the semispherical inner layer gel was included inside the outer layer gel immediately after loading the bright purple inner layer gel, but the boundary between the inner layer gel and the outer layer gel became unclear after 6 hours from loading. The semispherical shape of the inner gel could not be observed after 24 hours from loading.

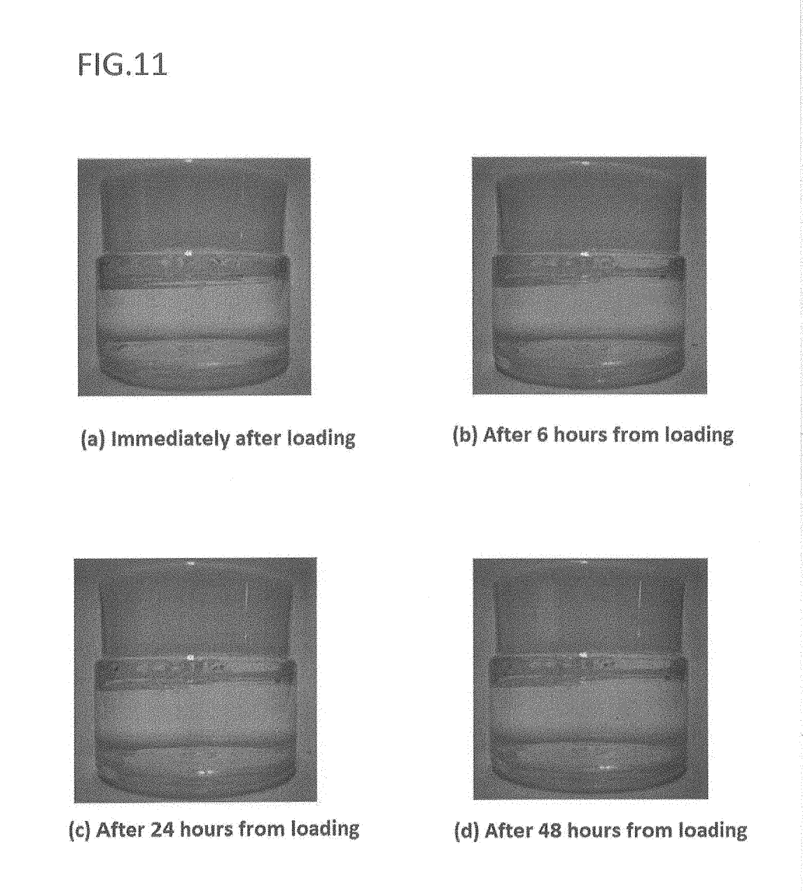

FIG. 11 shows photograph images showing the state of formulations for a product loaded with two formulations, in which the inner layer gel of prescription 11 is loaded into the outer layer gel of prescription 1, (a) immediately after loading, (b) after 6 hours from loading, (c) after 24 hours from loading, and (d) after 48 hours from loading the inner layer gel.

As can be understood from FIG. 11, in this product, the semispherical inner layer gel was included inside the outer layer gel immediately after loading the bright yellow green inner layer gel, but the boundary between the inner layer gel and the outer layer gel became unclear after 6 hours from loading. The inner layer gel and the outer layer gel were homogenously mixed after 24 hours from loading.

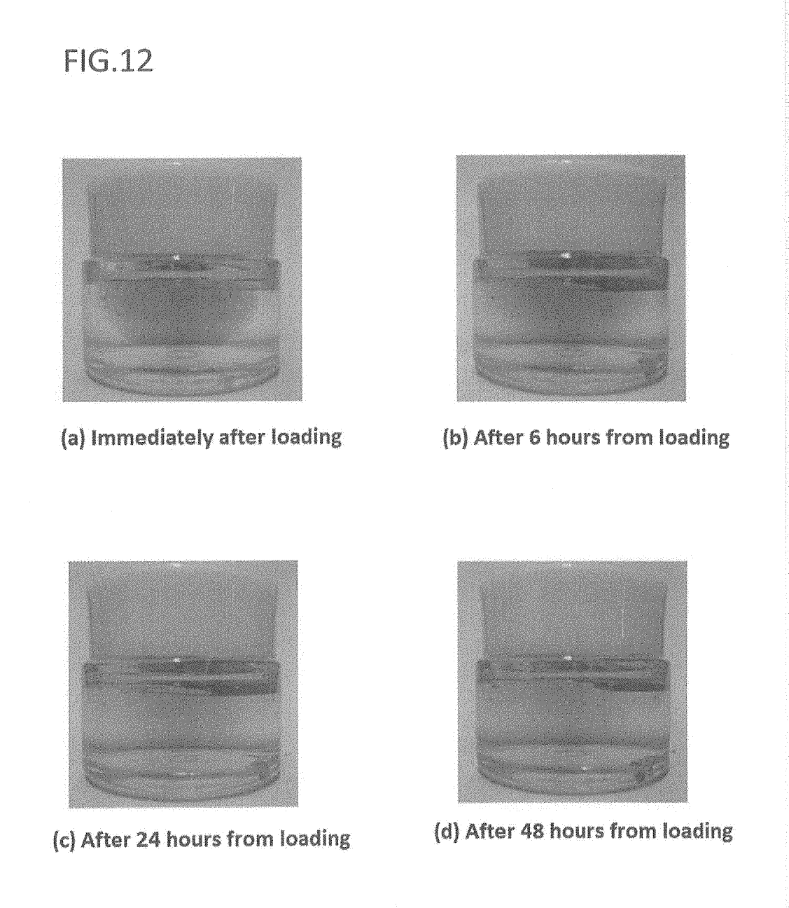

FIG. 12 shows photograph images showing the state of formulations for a product loaded with two formulations, in which the inner layer gel of prescription 12 is loaded into the outer layer gel of prescription 1, (a) immediately after loading, (b) after 6 hours from loading, (c) after 24 hours from loading, and (d) after 48 hours from loading the inner layer gel.

As can be understood from FIG. 12, in this product, the semispherical inner layer gel was included inside the outer layer gel immediately after loading the bright blue inner layer gel, but the boundary between the inner layer gel and the outer layer gel became unclear after 6 hours from loading. The semispherical shape of the inner gel could not be observed after 24 hours from loading.

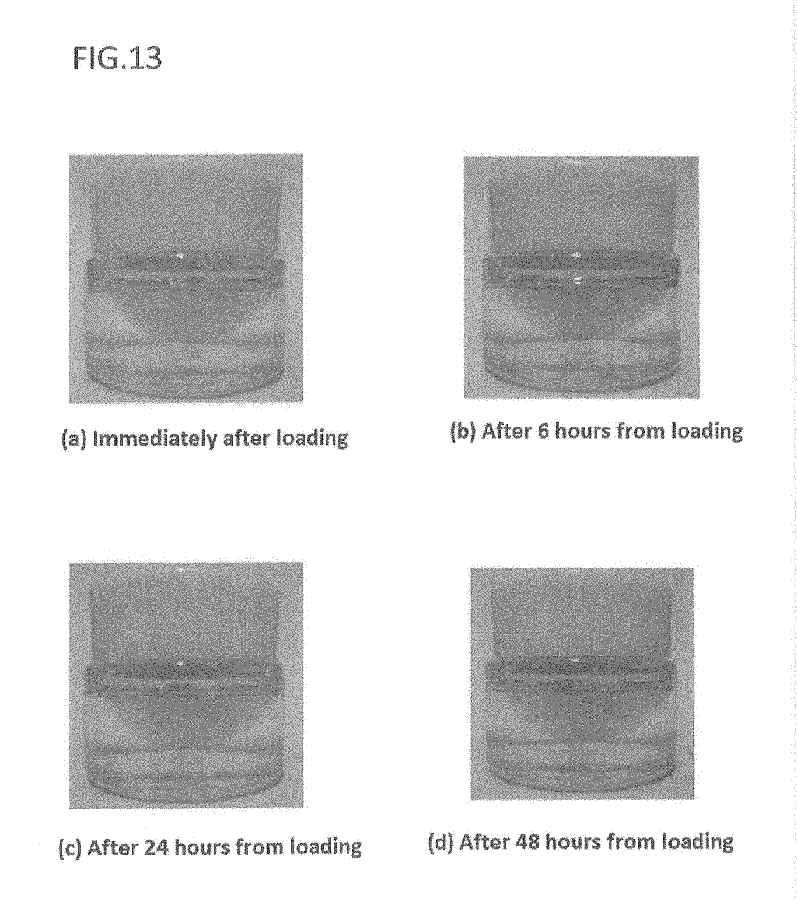

FIG. 13 shows photograph images showing the state of formulations for a product loaded with two formulations, in which the inner layer gel of prescription 13 is loaded into the outer layer gel of prescription 1, (a) immediately after loading, (b) after 6 hours from loading, (c) after 24 hours from loading, and (d) after 48 hours from loading the inner layer gel.

As can be understood from FIG. 13, in this product, the state in which semispherical bright yellow inner layer gel was included inside the outer layer gel was maintained from immediately after loading the inner layer gel to after 48 hours from loading. The boundary between the inner layer gel and the outer layer gel was clear even after 48 hours from loading.

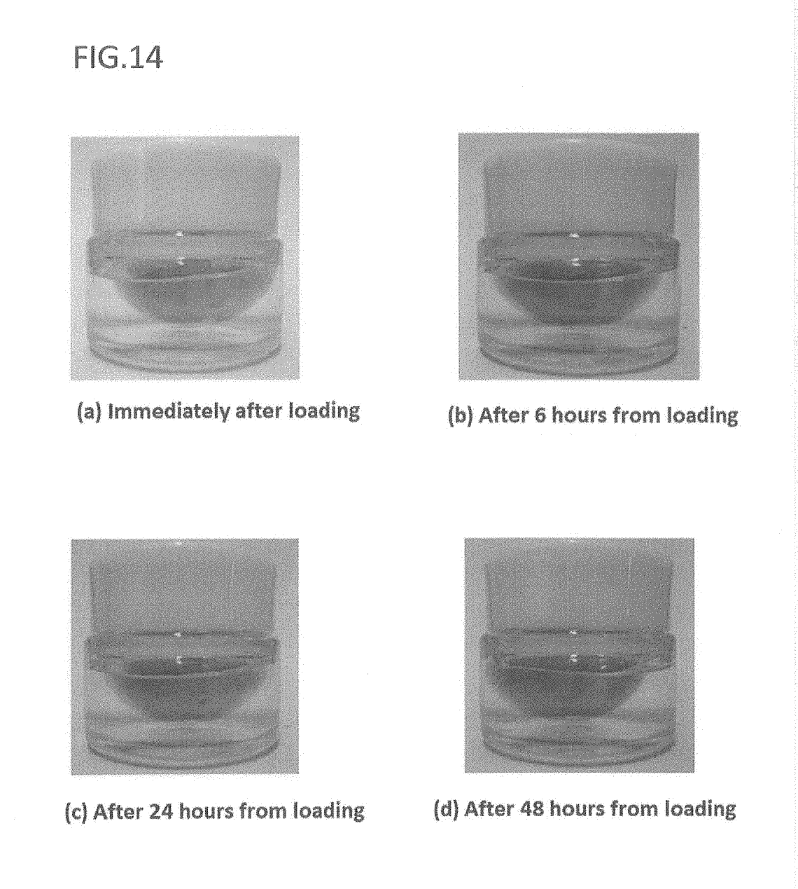

FIG. 14 shows photograph images showing the state of formulations for a product loaded with two formulations, in which the inner layer gel of prescription 14 is loaded into the outer layer gel of prescription 1, (a) immediately after loading, (b) after 6 hours from loading, (c) after 24 hours from loading, and (d) after 48 hours from loading the inner layer gel.

As can be understood from FIG. 14, in this product, the state in which semispherical bright purple inner layer gel was included inside the outer layer gel was maintained from immediately after loading the inner layer gel to after 48 hours from loading. The boundary between the inner layer gel and the outer layer gel was clear even after 48 hours from loading.

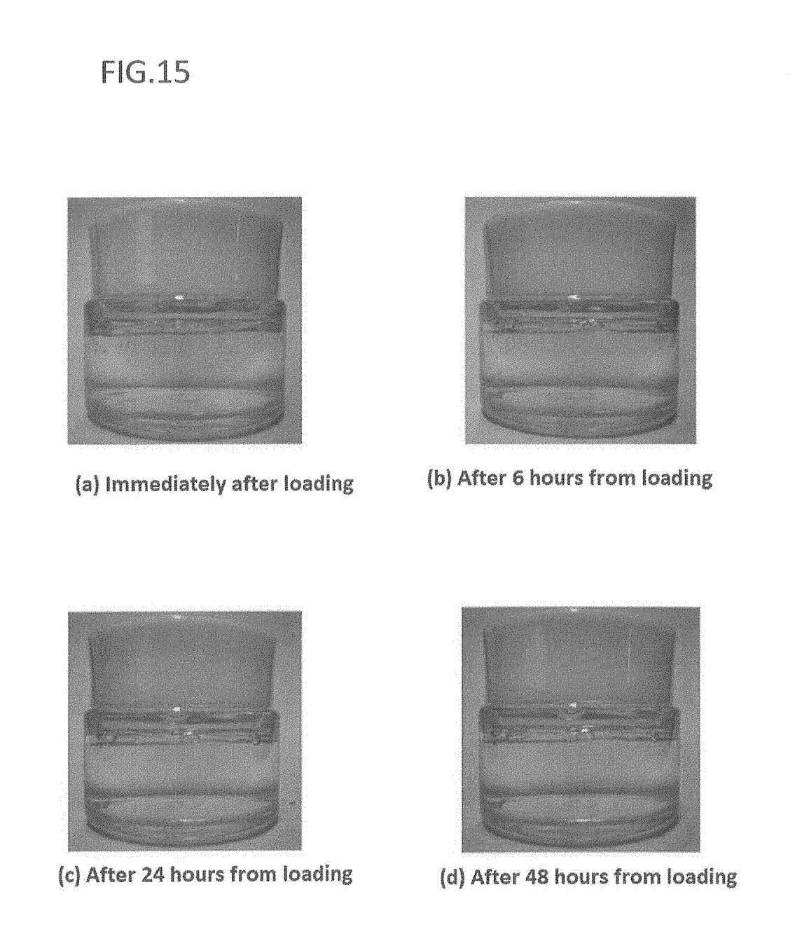

FIG. 15 shows photograph images showing the state of formulations for a product loaded with two formulations, in which the inner layer gel of prescription 15 is loaded into the outer layer gel of prescription 1, (a) immediately after loading, (b) after 6 hours from loading, (c) after 24 hours from loading, and (d) after 48 hours from loading the inner layer gel.

As can be understood from FIG. 15, in this product, the state in which semispherical bright yellow inner layer gel was included inside the outer layer gel was maintained from immediately after loading the inner layer gel to after 48 hours from loading. The boundary between the inner layer gel and the outer layer gel was clear even after 48 hours from loading.

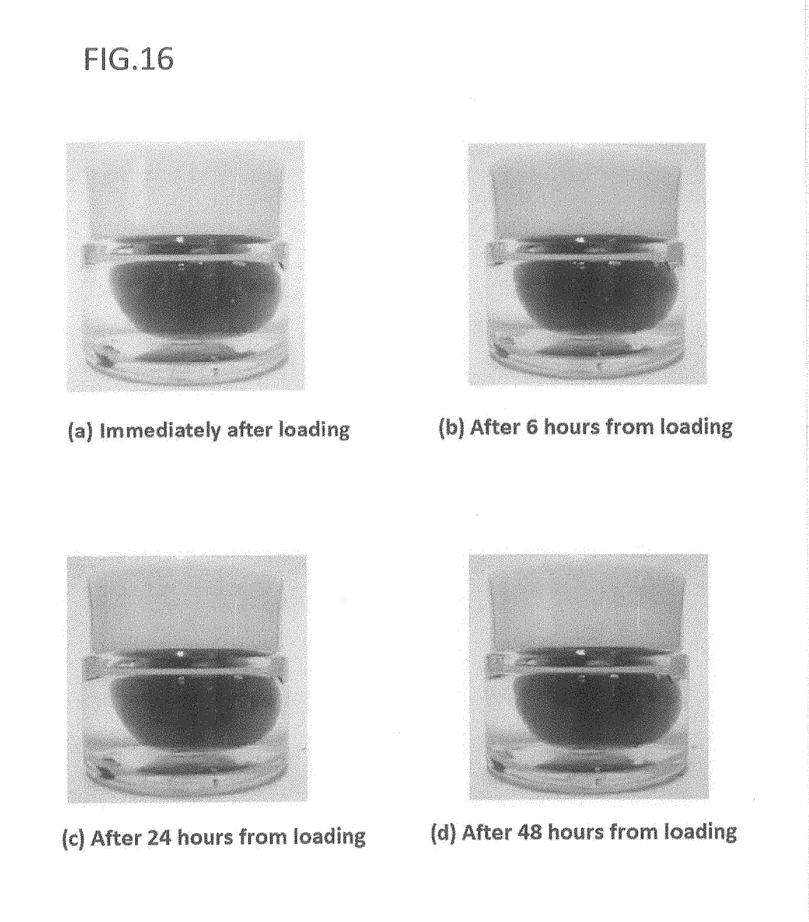

FIG. 16 shows photograph images showing the state of formulations for a product loaded with two formulations, in which the inner layer gel of prescription 16 is loaded into the outer layer gel of prescription 1, (a) immediately after loading, (b) after 6 hours from loading, (c) after 24 hours from loading, and (d) after 48 hours from loading the inner layer gel.

As can be understood from FIG. 16, in this product, the state in which semispherical black inner layer gel was included inside the outer layer gel was maintained from immediately after loading the inner layer gel to after 48 hours from loading. The boundary between the inner layer gel and the outer layer gel was clear even after 48 hours from loading.

In this manner, the inner layer gel dispersed into the outer layer gel with the passage of time after loading the inner layer gel such that a bilayer state could not be retained after 6 hours from loading in products loaded with two formulations using the inner gel of prescriptions 2 to 12. Meanwhile, the bilayer state was retained without the inner layer gel and the outer layer gel formulations mixing even after 48 hours from loading in the products loaded with two formulations using the inner gel of prescriptions 13 to 16 which used a colloid instead of a water-soluble stain as component (C). Especially when the gold colloid of prescription 14 was used, the amount of colloid added could be reduced further, and coloration of the inner layer gel from the gold colloid was superb. The product loaded with two formulations using the inner gel of prescription 16 had slightly inferior aesthetics relative to those using prescriptions 13 to 15 because the inner layer gel was opaque, but it was a product that could withstand practical use, with high stability of the bilayer state of the inner layer gel and the outer layer gel.

### <Evaluation of Functionality of Formulation>

Products loaded with two formulations using the inner layer gel of prescriptions 13 to 15 with superb capability to retain the shape of the formulation were used in usage trials to study the functionality of the formulations. In the usage trials, a user was instructed to take a suitable amount of formulation using a spatula and to apply the formulation to the skin. After use, the user was asked about the usability and functionality thereof in an interview format.

The products loaded with two formulations using the inner layer gel of prescriptions 13 to 15 comprise two types of formulations having the functionality of beauty gel comprising an aqueous component and moisturizing component such as glycerin or 1,3-butylene glycol in abundance. For this reason, when the product was taken with a spatula, including the formulations in both the inner layer portion and the outer layer portion, and applied to the skin, it was confirmed that a high humectifying effect, emollient effect, and moisturizing effect on the skin could be felt. It was also confirmed that the shape of the inner layer and the outer layer was maintained even after using a certain amount of the formulations, and the formulations can be used without impairing the aesthetics thereof even during use. In view of these results, it was demonstrated that a cosmetic product, which has functionalities of multiple formulations with different functionalities in the inner layer and the outer layer, and is capable of stably retaining a multilayer state where an inner layer formulation is included inside the outer layer formulation for a longer period of time, and has superb aesthetics, can be prepared by adding any cosmetic component to the inner layer formulation and/or outer layer formulation.

### INDUSTRIAL APPLICABILITY

The present invention can provide a cosmetic product, which is capable of stably retaining a multilayer state where the inner layer formulation is included inside the outer layer formulation for a longer period of time, even for formulations other than those using an emulsified composition as the inner layer and aqueous gel as the outer layer (e.g., formulations with the same dosage form). The cosmetic product of the present invention also has superb aesthetics and functionalities of multiple formulations in a single cosmetic product.

For example, the present invention can provide a cosmetic product with the following advantages.

(1) The present invention can provide a cosmetic product with functionalities of multiple formulations (preferably multiple formulations with the same dosage form) selected from skin care products such as aqueous gel, oily gel, beauty gel, aqueous cleansing gel, and oily cleansing gel in a single product.
(2) In one embodiment of the cosmetic product of the present invention, formulation A and formulation B with functionalities of two different types of cosmetic materials are combined while being separated. Since a component contained in one of the formulations do not have a significant effect on the other formulation, selectivity of components that can be added to each formulation is improved.
(3) In one embodiment of the cosmetic product of the present invention, two types of formulations with visually different shapes are combined while being separated in a transparent or semitransparent container and the inner formulation having a protruding curved surface at least on the surface thereof. A user can use the cosmetic product by visually adjusting the amount of two types of formulations used depending on the skin condition upon use or the like. In other words, a user can use the cosmetic product while making visual adjustments, such as using more of the formulation on the outside or more of the formulation on the inside depending on the skin condition. Despite being a single product, the product can be used differently according to the skin condition.
(4) A new type of cleansing formulation, which is capable of removing aqueous stains or makeup as well as oily stains or makeup, can be provided as a single product when aqueous cleansing gel is selected as one of the formulations and oily cleansing gel is selected as the other formulation and combined.
(5) In one embodiment of the cosmetic product of the present invention, a formulation in the inner layer has a shape that is completely enveloped by the formulation in the outer layer. Such a shape can dramatically reduce outflow of fragrant components such as fragrance or essential oil to the outside of the formulation up to immediately prior to use by adding the fragrant components only to the formulation in the inner layer portion. A cosmetic product of the present invention, which spreads the fresh scent as of immediately after the manufacture upon use of the cosmetic product, can be provided. Furthermore, the probability of being in directly contact with air is extremely low for a formulation in a shape that includes an oily component inside, so that oxidation of the oily component inside can be suppressed.
(6) A cosmetic product using highly transparent aqueous gel as the formulation in the outer layer and using colored gel as the formulation in the inner layer has better aesthetics. It is possible to provide a product with superb aesthetics and a mystical form in which a spherical gel layer is completely surrounded by gel, depending on the shape of the container, ratio of the aqueous gel in the outer layer and the formulation in the inner layer that are added, and the presence/absence of color.

As discussed above, the present invention can provide a cosmetic product with various functionalities and aesthetics that existing products do not have.

### REFERENCE SIGNS LIST

1 Cosmetic product
2 Container
A Formulation in the outer layer
B Formulation in the inner layer

1. A cosmetic product having a transparent or semitransparent container, a transparent or semitransparent formulation A loaded into the container, and a formulation B embedded in the formulation A,
wherein at least one of the formulations A and B comprise a colloid.

2. The cosmetic product of claim 1, wherein the formulation A and the formulation B have the same dosage form.

3. The cosmetic product of claim 2, wherein the dosage form of the formulation A and the formulation B is aqueous gel.

4. The cosmetic product of claim 1, wherein the formulation B is transparent or semitransparent.

5. The cosmetic product of claim 1, wherein the formulation B comprises a colloid, and an amount of a colloid dispersion added relative to the entire amount of the formulation B is 0.001 to 50.00% by weight.

6. The cosmetic product of claim 1, wherein an average particle size of the colloid is 1 nm to 10 μm.

7. The cosmetic product of claim 1, wherein the colloid is a metal colloid.

8. The cosmetic product of claim 7, wherein the metal colloid is a metal colloid selected from the group consisting of platinum, gold, silver, palladium, rhodium, ruthenium, iridium, and a combination thereof, and an alloy prepared by mixing these metals.

9. The cosmetic product of claim 1, wherein a maximum load indicating a viscosity of the formulation A and the formulation B measured under the following conditions is 0.2 N to 2.0 N:
a maximum value of load applied per cross-sectional area of a rod-like probe with a diameter of 20 mm when the rod-like probe is inserted 25 mm at a rate of 60 mm/min as measured with Sun Scientific Co., Ltd.'s SUN RHEO METER (COMPAC 100-II).

10. The cosmetic product of claim 1, wherein a ratio of the formulation B to the entire amount of a formulation is 10 to 60% by weight.

11. The cosmetic product of claim 1, wherein a shape of the formulation B is a shape selected from the group consisting of a perfectly spherical shape, a spherical shape, an egg shape, a spheroid shape, a shape with an oval cross-section, a perfectly spherical shape with a protrusion on a surface, a spherical shape with a protrusion on a surface, an egg shape with a protrusion on a surface, a spheroid shape with a protrusion on a surface, and a shape with an oval cross-section with a protrusion on a surface.
